(19) United States
(12) Patent Application Publication
(10) Pub. No.: US 2020/0121748 A1
(43) Pub. Date: Apr. 23, 2020

(54) CANNABIS FORMULATION AND METHOD OF PREPARATION

(71) Applicants: TOMMY JOHN GUERRAZZI, LOMA MAR, CA (US); DORIAN ORION GUERRAZZI, LOMA MAR, CA (US)

(72) Inventors: TOMMY JOHN GUERRAZZI, LOMA MAR, CA (US); DORIAN ORION GUERRAZZI, LOMA MAR, CA (US)

(73) Assignee: Purity AgriTech Corp., MEDICINE HAT (CA)

(21) Appl. No.: 16/159,438

(22) Filed: Oct. 12, 2018

(51) Int. Cl. A61K 36/185; A61K 31/352; A61K 31/05; A61K 47/24; A61K 47/36; A61K 47/22

(52) U.S. Cl. CPC A61K 36/185; A61K 31/352; A61K 31/05; A61K 2236/39; A61K 47/36; A61K 47/22; A61K 47/24

(57) ABSTRACT

Cannabis formulations and method of preparing the cannabis formulations. A phytocannabinoid preparation, such as an extract or distillate, is heated to a setpoint temperature at which the phytocannabinoid preparation is flowable. The setpoint may be between 90 and 105° F. A carrier oil is combined with the phytocannabinoid preparation. High-shear mixing is applied to the carrier oil and the phytocannabinoid preparation to provide a diluted preparation. Lecithin is combined with the diluted preparation and additional high-shear mixing is applied the lecithin with the diluted preparation, providing a cannabis formulation. The cannabis formulation may be cavitated with ultrasonic mixing. The cannabis formulation may be combined with maltodextrin to provide a solid phytocannabinoid formulation.

### FIELD

The present disclosure relates to cannabis formulations and methods for preparing the cannabis formulations.

### BACKGROUND

Many jurisdictions within the United States and worldwide are moving from prohibition of cannabis to regulation of cannabis. Cannabis is being separately regulated for medical or adult use markets. Medical, general health, and other benefits of cannabis are increasingly recognized. The variety of products being prepared that include cannabis is expanding.

Dried cannabis is considered by many to be the standard form of cannabis and is suitable for smoking or vaporizing. However, cannabis products other than dried cannabis flower have gained increasing market share in regulated markets. Phytocannabinoids and other chemicals found cannabis resin may be extracted from Cannabis sativa biomass using solvents or solventless methods. Cannabis extract may be formulated into a wide variety of products that are suitable for eating, drinking, topical application, transmucosal absorption or vaporizing.

### SUMMARY

Many jurisdictions are moving from prohibition to regulation of cannabis, resulting in a greater market for cannabis in a regulated environment. With more applications and an interest in controlled dosing, there is a need for cannabis formulations that facilitate consistent and predictable dosing, and that are reasonably shelf-stable. Phytocannabinoids are lipophilic, which defines many of the challenges associated with formulating phytocannabinoid extracts, pressed resins, distillate or other phytocannabinoid preparations into user-friendly and bioavailable dosage forms. Formulations described herein may be prepared from phytocannabinoid preparations to facilitate consistent and accurate dosing.

Generally, the present method is applied to a phytocannabinoid preparation. The phytocannabinoid preparation may include phytocannabinoids, terpenoids, phenylpropanoids and other compounds recovered from cannabis plant matter. The phytocannabinoid preparation may be extracted from cannabis plant matter by a variety of techniques using suitable solvents (e.g. CO₂, alcohols, hydrocarbons, etc.), or through solventless methods (e.g. pressing, etc.). The extract may be winterized or otherwise subject to midstream processing. The phytocannabinoid preparation may be referred to generally as extract, crude oil, resin, rosin, wax, distillate or by other names depending on the specific manner in which the phytocannabinoid preparation is prepared from trichome-bearing Cannabis sativa biomass.

The phytocannabinoid preparation is heated to a setpoint temperature where it is between 1,500 and 2,000 centipoise, and flowable. The setpoint temperature may be between 90 and 105° F. A carrier oil is added to reduce the dynamic viscosity of the phytocannabinoid preparation. The carrier oil may be coconut oil with melting onset of 76° F., coconut oil with a melting onset of 92° F., olive oil, grapeseed oil, or any suitable oil. The carrier oil may be warmed to the setpoint temperature before being added to the phytocannabinoids preparation. The carrier oil may be added in roughly equal parts volume to the phytocannabinoid preparation.

High-shear mixing is applied to homogenize the phytocannabinoid preparation with the carrier oil while maintaining the setpoint temperature, providing a diluted preparation. A mechanical high-shear mixer, such as a rotor-stator mixer or other high-shear mixer may be applied to disrupt associations between molecules.

After the carrier oil is homogenized with the phytocannabinoid preparation, lecithin is combined with the resulting diluted preparation and the high-shear mixing continues. An amount of liquid lecithin equal to about 50% of the volume of the diluted preparation is added to the diluted preparation. The setpoint temperature may be maintained while combining the lecithin with the diluted preparation, such as through use of a warm water bath during mixing, to provide a cannabis formulation.

The cannabis formulation may be added to an ultrasonic homogenizer and exposed to high-intensity sonic waves to introduce cavitation in the cannabis formulation and provide a cavitated cannabis formulation.

During high-shear mixing or during the ultrasonic homogenization, about 2 to 3 percent of the sample size in vitamin E solution may be added to the cannabis formulation, and included in the cannabis formulation for mitigating rancidity and extending the shelf life of the cannabis formulation. Other antioxidants may be used in place of the vitamin E.

The cannabis formulation may be combined with carrier oil and glycerin to dilute the cannabis formulation to a defined dosage of phytocannabinoids that allows simple dosing either by encapsulating in gel caps or soft gels, or through a syringe or dropper.

The cannabis formulation may be combined with maltodextrin to prepare a solid cannabis formulation for transmucosal or edible use. An additional carrier oil may be added to the cannabis formulation to calibrate the dosage of the solid cannabis formulation, such as about 10 mg of phytocannabinoids per gram of solid cannabis formulation. The additional carrier oil may be the same carrier oil that was added to the phytocannabinoid preparation or any other suitable oil. During and after combining the cannabis formulation with the maltodextrin, the resulting mixture may be maintained at about 80 to 85° F. The cannabis formulation may be heated to 80 to 85° F. and then poured over the maltodextrin, followed by mixing. The maltodextrin may be preheated to dehydrate the maltodextrin prior to being mixed with the cannabis formulation.

The method may be practiced under a modified local atmosphere by pumping oxygen and water out of a chamber and pumping nitrogen into the chamber, displacing the oxygen and water. The modified local atmosphere may mitigate rancidity due to lowered oxygen and mitigate bacterial proliferation due to lowered humidity.

In a first aspect, herein provided are cannabis formulations and method of preparing the cannabis formulations. A phytocannabinoid preparation, such as an extract or distillate, is heated to a setpoint temperature at which the phytocannabinoid preparation is flowable. The setpoint may be between 90 and 105° F. A carrier oil is combined with the phytocannabinoid preparation. High-shear mixing is applied to the carrier oil and the phytocannabinoid preparation to provide a diluted preparation. Lecithin is combined with the diluted preparation and additional high-shear mixing is applied the lecithin with the diluted preparation, providing a cannabis formulation. The cannabis formulation may be cavitated with ultrasonic mixing. The cannabis formulation may be combined with maltodextrin to provide a solid phytocannabinoid formulation

In a further aspect, herein provided is a method of preparing a cannabis formulation comprising: providing a phytocannabinoid preparation; heating the phytocannabinoid preparation to a setpoint temperature at which the phytocannabinoid preparation is flowable; combining a carrier oil with the phytocannabinoid preparation; high-shear mixing the carrier oil with the phytocannabinoid preparation to provide a diluted preparation; combining lecithin with the diluted preparation; and high-shear mixing the lecithin with the diluted preparation, providing the cannabis formulation.

In some embodiments, the phytocannabinoid preparation comprises a phytocannabinoid distillate.

In some embodiments, the phytocannabinoid preparation comprises terpenoids.

In some embodiments, the phytocannabinoid preparation comprises phytocannabinoids and terpenoids extracted from a single biomass source.

In some embodiments, wherein the single biomass source comprises plant matter from a single clonal strain or stable variety of Cannabis sativa.

In some embodiments, the setpoint temperature is between 90 and 105° F.

In some embodiments, the cannabis preparation has a dynamic viscosity of between 1,500 and 2,000 centipoise at the setpoint temperature.

In some embodiments, combining the carrier oil with the cannabis preparation comprises heating the carrier oil to the setpoint temperature.

In some embodiments, high-shear mixing the carrier oil with the phytocannabinoid preparation comprises high-shear mixing with a rotor-stator mixer.

In some embodiments, high-shear mixing the carrier oil with the phytocannabinoid preparation comprises mixing at the setpoint temperature.

In some embodiments, combining lecithin with the diluted preparation comprises heating the lecithin to the setpoint temperature

In some embodiments, high-shear mixing the lecithin with the diluted preparation comprises heating the lecithin to a lecithin setpoint temperature.

In some embodiments, high-shear mixing the lecithin with the diluted preparation comprises high-shear mixing with a rotor-stator mixer.

In some embodiments, the lecithin comprises sunflower lecithin.

In some embodiments, the method includes applying ultrasonic homogenization to the cannabis formulation, providing a cavitated cannabis formulation.

In some embodiments, the method includes combining maltodextrin with the cannabis formulation, providing a solid cannabis formulation.

In some embodiments, combining maltodextrin with the cannabis formulation comprises maintaining the cannabis formulation at a mixing temperature at which the cannabis formulation has a dynamic viscosity of between 150 and 400 centipoise.

In some embodiments, the mixing temperature is between 80 and 85 degrees Fahrenheit.

In some embodiments, combining maltodextrin with the cannabis formulation comprises adding an additional carrier oil to the cannabis.

In some embodiments, the method includes dehydrating the maltodextrin.

In some embodiments, the method includes combining an antioxidant with the cannabis formulation.

In some embodiments, the antioxidant comprises five percent or less by volume in the cannabis formulation Vitamin E.

In some embodiments, the method includes combining an additional carrier oil to the cannabis formulation, targeting a phytocannabinoid concentration to provide a calibrated liquid cannabis formulation.

In some embodiments, providing the phytocannabinoid preparation, heating the phytocannabinoid preparation to the setpoint temperature, combining the carrier oil with the phytocannabinoid preparation, high-shear mixing the carrier oil with the phytocannabinoid preparation, combining lecithin with the diluted preparation, and high-shear mixing the lecithin with the diluted preparation are performed in a nitrogen-enriched atmosphere for dehydrating the phytocannabinoid preparation and mitigating oxidative damage.

In some embodiments, the method includes packaging the phytocannabinoid preparation in the nitrogen-enriched atmosphere.

In some embodiments, the method includes combining maltodextrin with the cannabis formulation in the nitrogen-enriched atmosphere, providing a solid cannabis formulation.

In some embodiments, providing the phytocannabinoid preparation, heating the phytocannabinoid preparation to the setpoint temperature, combining the carrier oil with the phytocannabinoid preparation, high-shear mixing the carrier oil with the phytocannabinoid preparation, combining lecithin with the diluted preparation, and high-shear mixing the lecithin with the diluted preparation are performed in a argon-enriched atmosphere for dehydrating the phytocannabinoid preparation and mitigating oxidative damage.

In some embodiments, the method includes packaging the phytocannabinoid preparation in the argon-enriched atmosphere.

In some embodiments, the method includes combining maltodextrin with the cannabis formulation in the argon-enriched atmosphere, providing a solid cannabis formulation.

In a further aspect, herein provided is a method of preparing a phytocannabinoid formulation comprising: providing a phytocannabinoid preparation; heating the phytocannabinoid preparation to a setpoint temperature at which the phytocannabinoid preparation has a dynamic viscosity of between 1,500 and 2,000 centipoise; combining a carrier oil with the phytocannabinoid preparation; high-shear mixing the carrier oil with the phytocannabinoid preparation at or above the setpoint temperature to provide a diluted preparation; heating lecithin to provide liquid lecithin; combining the liquid lecithin with the diluted preparation; high-shear mixing the liquid lecithin with the diluted preparation, providing the cannabis formulation; and applying ultrasonic homogenization to the cannabis formulation, providing cavitated cannabis formulation.

In some embodiments, the method includes combining maltodextrin with the cannabis formulation, providing a solid cannabis formulation.

In some embodiments, combining maltodextrin with the cannabis formulation comprises maintaining the cannabis formulation at a mixing temperature at which the cannabis formulation has a dynamic viscosity of between 150 and 400 centipoise.

In some embodiments, the mixing temperature is between 80 and 85 degrees Fahrenheit.

In some embodiments, combining maltodextrin with the cannabis formulation comprises adding an additional carrier oil to the cannabis.

In some embodiments, the method includes dehydrating the maltodextrin.

In a further aspect, herein provided is a cannabis formulation according to any method described herein.

Other aspects and features of the present disclosure will become apparent to those ordinarily skilled in the art upon review of the following description of specific embodiments in conjunction with the accompanying figures.

### BRIEF DESCRIPTION OF THE DRAWINGS

Embodiments of the present disclosure will now be described, by way of example only, with reference to the attached figures, in which reference numerals sharing a common final two digits refer to corresponding features across figures (e.g. heating the phytocannabinoid preparation 20, 120, 220, 320, 420, 520, etc.):

FIG. 1 is a flow chart of a method of preparing a cannabis formulation;

FIG. 2 is a flow chart of a method of preparing a cannabis formulation;

FIG. 3 is a flow chart of a method of preparing a cannabis formulation;

FIG. 4 is a flow chart of a method of preparing a cannabis formulation;

FIG. 5 is a flow chart of a method of preparing a cannabis formulation; and

FIG. 6 is a flow chart of a method of preparing a cannabis formulation.

### DETAILED DESCRIPTION

Generally, the present disclosure provides cannabis formulations and methods of preparing the cannabis formulation. The formulations described herein may provide advantages over smoking and vaping cannabis, including facilitating dosing consistency, efficiency, and avoiding inconvenience, social consequences and health consequences associated with smoking and vaping.

Many jurisdictions are moving from prohibition to regulation of Cannabis sativa, including varieties that do not meet the criteria of industrial hemp. As used herein, C. sativa refers to a single plant species that includes at least three major subspecies, which are C. sativa sativa, C. sativa indica, and C. sativa ruderalis, and includes all landraces, hybrids or other varieties, cultivars or clonal strains defined by or derived from any of these subspecies, including any industrial hemp or drug-type varieties, cultivars or clonal strains. Cannabis is a blanket term for dried or fresh cannabis flowers (or other plant matter), and any products prepared from extracts of C. sativa or that include phytocannabinoids identical to naturally occurring phytocannabinoids regardless of source.

Cannabis products come in many forms and include one or more phytocannabinoids. As used herein, “phytocannabinoid” or “phytocannabinoids” may include any phytocannabinoid regardless of source (C. sativa derived, biosynthesized in other organisms, structurally identical synthetic), that includes one or more of delta-9-tetrahydrocannabinol (“THC”), cannabidiol (“CBD”), cannabichromene (“CBC”), cannabielsoin (“CBE”), delta-8-tetrahydrocannabinol (“Δ8-THC”), iso-tetrahydrocannabinol (“iso-THC”), cannabicyclol (“CBL”), cannabicitran (“CBT”), any acid form (i.e. not decarboxylated), any varin side chain form (e.g. THCv, CBDv, etc.), any chemically modified phytocannabinoid (e.g. THC acetate, etc.), any other phytocannabinoid produced by any variety of C. sativa or that are known degradation products that have psychoactive or other properties (e.g. 11-hydroxy-THC, cannabinol (“CBN”), etc.).

The global trend away from prohibition and toward regulation has resulted in a greater market for regulated cannabis products. Greater variety in phytocannabinoids, a better understanding of the entourage effect between phytocannabinoids and terpenoids, phenylpropanoids and other chemicals, there is an increased demand for controlled dosing in both medical and adult use markets. There is therefore a need for cannabis products formulated to facilitate consistent and predictable dosing, and that are with improved shelf stability. Phytocannabinoids are lipophilic, which defines many of the challenges associated with formulating phytocannabinoid extracts, pressed resins, distillate or other phytocannabinoid preparations into user-friendly and bioavailable dosage forms. Formulations and methods of preparing the same as described herein may be prepared from phytocannabinoid preparations to facilitate consistent and accurate dosing.

The methods described herein may be applied to preparing a phytocannabinoids formulation from a phytocannabinoid preparation. The phytocannabinoid preparation may include phytocannabinoids, terpenoids, phenylpropanoids and other compounds recovered from C. sativa plant matter. The phytocannabinoid preparation may be extracted from C. sativa plant matter by a variety of techniques using suitable solvents (e.g. CO₂, alcohols, hydrocarbons, etc.), or through solventless methods (e.g. pressing, etc.).

The extract may be winterized or otherwise subject to midstream processing. The phytocannabinoid preparation may be referred to generally as extract, crude oil, resin, rosin, wax, distillate or by other names depending on the specific manner in which the phytocannabinoid preparation is prepared from trichome-bearing C. sativa biomass.

The phytocannabinoid preparation is heated to a setpoint temperature. At the setpoint temperature, the phytocannabinoid preparation may have a dynamic viscosity of between 1,500 and 2,000 centipoise. At a dynamic viscosity in this range, the phytocannabinoid preparation is flowable. The setpoint temperature may be between 90 and 105° F. Temperature control may be accomplished using a hot water bath and heat exchange piping.

A carrier oil is added to the phytocannabinoid preparation to reduce the dynamic viscosity of the phytocannabinoid preparation in a resulting diluted preparation. The carrier oil may have a dynamic viscosity of about 100 centipoise at 70° F. The carrier oil may be added before or after heating the phytocannabinoid preparation to the setpoint temperature.

The carrier oil may be any suitable food oil, such as coconut oil with melting onset of 76° F., coconut oil with a melting onset of 92° F., olive oil, grapeseed oil, or any suitable oil. The carrier oil may be warmed to the setpoint temperature before being added to the phytocannabinoids preparation. The carrier oil may be added in roughly equal parts volume to the phytocannabinoid preparation. The carrier oil thins out the phytocannabinoid preparation, facilitating mechanical processing of the diluted preparation resulting from high-shear mixing compared with the phytocannabinoid preparation.

High-shear mixing is applied to the phytocannabinoid preparation and the carrier oil while maintaining the setpoint temperature to homogenize the phytocannabinoid preparation with the carrier oil, providing a diluted preparation. A mechanical high-shear mixer, such as a rotor-stator mixer or other high-shear mixer may be applied to disrupt associations between molecules. High-shear mixing of a fluid applies one or more impellers or rotors that work the fluid. Rotational velocity is greater at the tip of the impeller or rotor than at the base of the rotor. The difference in velocity creates shear as fluid move at different speeds relative to one another. A stator may be included around the impeller or rotor. The interface between the stator and the tip of the rotor results in an extremely high-shear.

High-shear mixing may be applied to the phytocannabinoid preparation and the carrier oil to provide the diluted preparation. Suitable high-shear mixers may include rotor-stator mixers. At small scale, a pro Scientific Bio-Gen PRO200 hand held homogenizer with a ⅕ HP motor and a range of 5,000 to 35,000 RPM may be applied. The PRO200 may be used with rotor stators ranging in size from 5mm to 20mm for batch sizes from 0.03 ml all the way up to a full liter. A Pro Scientific PRO200 10 mm×115 mm homogenizer rotor stator may be used for high-shear mixing.

Physical shearing action in the presence of oleic or linoleic, which are abundant in most carrier oils may facilitate intermolecular separation of THC, CBD and other phytocannabinoids by disrupting interactions between these molecules. This separation may facilitate interaction between the lecithin and the phytocannabinoids to bind to each other. The ultrasonic cavitation may facilitate nanoscale interactions between the lecithin and the phytocannabinoids.

After the carrier oil is homogenized with the phytocannabinoid preparation, lecithin is combined with the diluted preparation. In some cases, A ratio of 1:1:1 of phytocannabinoid preparation:carrier oil:lecithin by volume may be used. In other cases, an amount of lecithin equal to about 50% of the volume of the diluted preparation is added to the diluted preparation to provide a cannabis formulation. The setpoint temperature may be maintained while combining the lecithin with the diluted preparation, such as through use of a warm water bath during mixing. The lecithin may be heated to the point where it is liquid flowable, which may facilitate mixing with the diluted preparation compared with dissolving solid lecithin into the diluted preparation. The lecithin may be heated to the setpoint temperature. The lecithin may provide a formulation with some similarity to a food matrix and potentially facilitate absorption.

The lecithin includes one or more phospholipids, which each have hydrophobic and hydrophilic groups. The lecithin may include Phosphatidylinositol, Phosphatidylcholine, and Phosphatidylethanolamine. Lecithin is used in cooking and drug formulation as an emulsification agent. Other liquid and solid emulsifying agents may be applied with changes to duration temperature of different operations. Some emulsifying agents may be better suited to specific naturally-occurring fats, lipids and other lipophilic compounds that may have different ratios in different varieties of C. sativa.

After the lecithin is added to the diluted preparation, the high-shear mixing continues. This continued high-shear mixing may be at the setpoint temperature The cannabis formulation may be added to an ultrasonic homogenizer and exposed to high-intensity sonic waves to introduce cavitation in the cannabis formulation and provide a cavitated cannabis formulation. The cannabis formulation or the cavitated cannabis formulation may be packaged and stored for use as concentrated phytocannabinoids or as inputs in skin care products, edible cannabis products or other cannabis products.

The ultrasonic homogenizer uses high intensity, high-frequency sound waves to induce cavitation in a liquid. The high-frequency sound waves may be about 20 kHz. Cavitation occurs as vacuum-bubbles or voids form and then collapse, disrupting any plant tissue in the phytocannabinoid formulation. Cavitation supports emulsification and nanoparticle dispersion of the phytocannabinoid formulation after high-speed mechanical shearing. In addition, sonochemistry may facilitate prolonging the shelf life of the cannabis formulation with addition of DL-Alpha-Tocopheryl Acetate, other Vitamin E preparations or other anti-oxidants during ultrasonic homogenization or high-shear mixing. A 900 w Digital Sonicator operating in the 20 KHz frequency range with an integrated sound abatement chamber to reduce cavitational sound emitted during run times may be applied to test batches of between 20 ml and 1,000 ml.

During high-shear mixing or during the ultrasonic homogenization, a vitamin E solution may be added to the cannabis formulation. The vitamin E solution may be added in an amount equal to about 5 percent, in some cases between 2 and 3 percent, of the sample size. The vitamin E solution may mitigate rancidity and extending the shelf life of the cannabis formulation. The vitamin E solution may be DL-Alpha-Tocopheryl Acetate specifically.

The cannabis formulation may be combined with carrier oil and glycerin to dilute the cannabis formulation to a defined dosage of phytocannabinoids that allows simple dosing either by encapsulating in gel caps or soft gels, or through a syringe or dropper.

The cannabis formulation may be combined with maltodextrin to prepare a solid cannabis formulation for transmucosal or edible use. An additional carrier oil may be added to the cannabis formulation to calibrate the dosage of the solid cannabis formulation, such as about 10 mg of phytocannabinoids per gram of solid cannabis formulation. After the maltodextrin is added to the cannabis formulation, a planetary gear mixer or other low-power mixer is applied to mix the maltodextrin with the cannabis formulation. The solid cannabis formulation may be pressed into a lozenge, dried and crumbled into a powder, combined with a food matrix or otherwise prepared for transmucosal use or swallowing.

The additional carrier oil may be the same carrier oil that was added to the phytocannabinoid preparation or any other suitable oil. During and after combining the cannabis formulation with the maltodextrin, the resulting mixture may be maintained at a temperature at which the cannabis formulation has a dynamic viscosity of between 150 and 200 centipoise, or below 150 centipoise, which may be at about 80 to 85° F. The cannabis formulation may be heated to 80 to 85° F. and then poured over the maltodextrin, followed by mixing. The maltodextrin may be preheated to between 100 and 110° F. to dehydrate the maltodextrin prior to mixing the maltodextrin with the cannabis formulation. Blood orange, mint or other flavours and additives may be added to the maltodextrin for flavour or for potential additional benefits.

The method may be carried out under a desiccated nitrogen or argon atmosphere to mitigate oxidation and moisture contamination. This may reduce rancidity by the absence or low abundance of oxygen and reduce proliferation of bacteria or mold by the absence or low abundance of humidity. The phytocannabinoid preparation, the carrier oil, the lecithin and if applicable the antioxidant are placed in vacuum tanks and placed under vacuum to remove water and oxygen. Once the vacuum is applied, nitrogen, argon or another inert gas is pumped into the vacuum tanks. A sealed workstation is similarly purged of oxygen and water under vacuum and filled with nitrogen, argon or another inert gas.

Providing the phytocannabinoid preparation, heating the phytocannabinoid preparation to the setpoint temperature, combining the carrier oil with the phytocannabinoid preparation, high-shear mixing the carrier oil with the phytocannabinoid preparation, combining lecithin with the diluted preparation, and high-shear mixing the lecithin with the diluted preparation are performed in the sealed workstation. Combining maltodextrin with the cannabis formulation, diluting the cannabis formulation and packaging the resulting solid cannabis formulation or dose-calibrated cannabis.

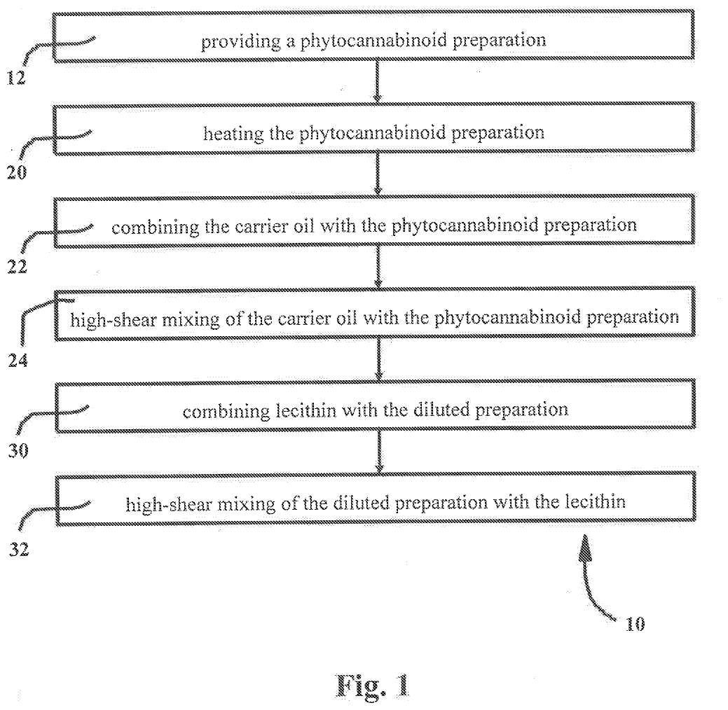

FIG. 1 shows a method of preparing a phytocannabinoid formulation 10. The method 10 includes providing a phytocannabinoid preparation 12. Heating the phytocannabinoid preparation 20 follows until the phytocannabinoid preparation is flowable. After heating the phytocannabinoid preparation 20, combining the carrier oil with the phytocannabinoid preparation 22 follows. After combining the carrier oil with the phytocannabinoid preparation 22, high-shear mixing of the carrier oil with the phytocannabinoid preparation 24 follows until the diluted preparation is produced by the high-shear mixing. Combining lecithin with the diluted preparation 30 follows preparation of the diluted preparation. High-shear mixing 32 of the diluted preparation with the lecithin follows to prepare the cannabis formulation.

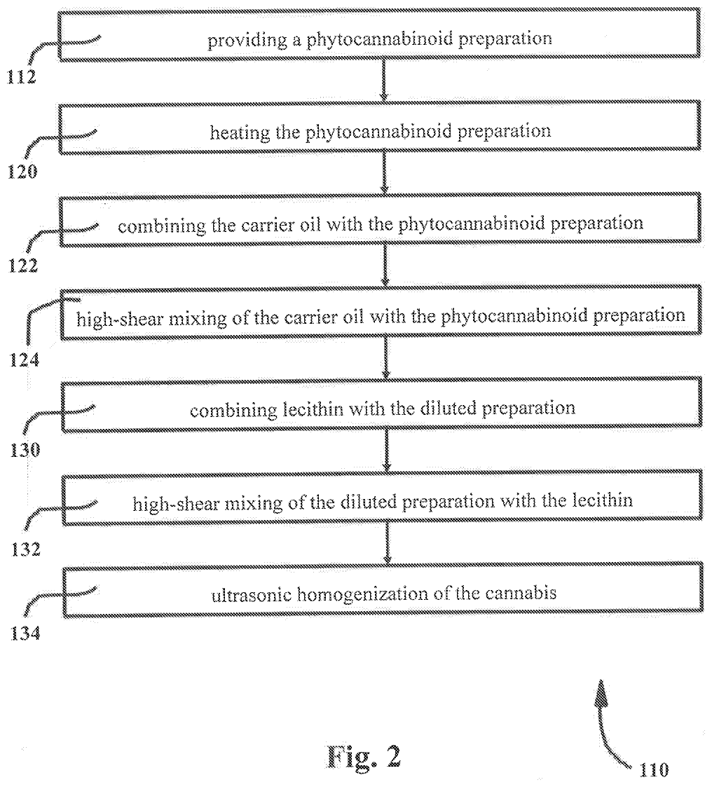

FIG. 2 shows a method of preparing a phytocannabinoid formulation 110. The method 110 includes providing a phytocannabinoid preparation 112. Heating the phytocannabinoid preparation 120 follows until the phytocannabinoid preparation is flowable. After heating the phytocannabinoid preparation 120, combining the carrier oil with the phytocannabinoid preparation 122 follows. After combining the carrier oil with the phytocannabinoid preparation 122, high-shear mixing of the carrier oil with the phytocannabinoid preparation 124 follows until the diluted preparation is produced by the high-shear mixing. Combining lecithin with the diluted preparation 130 follows preparation of the diluted preparation. High-shear mixing 132 of the diluted preparation with the lecithin follows to prepare the cannabis formulation. Ultrasonic homogenization of the cannabis formulation 134 follows, resulting in the cavitated formulation.

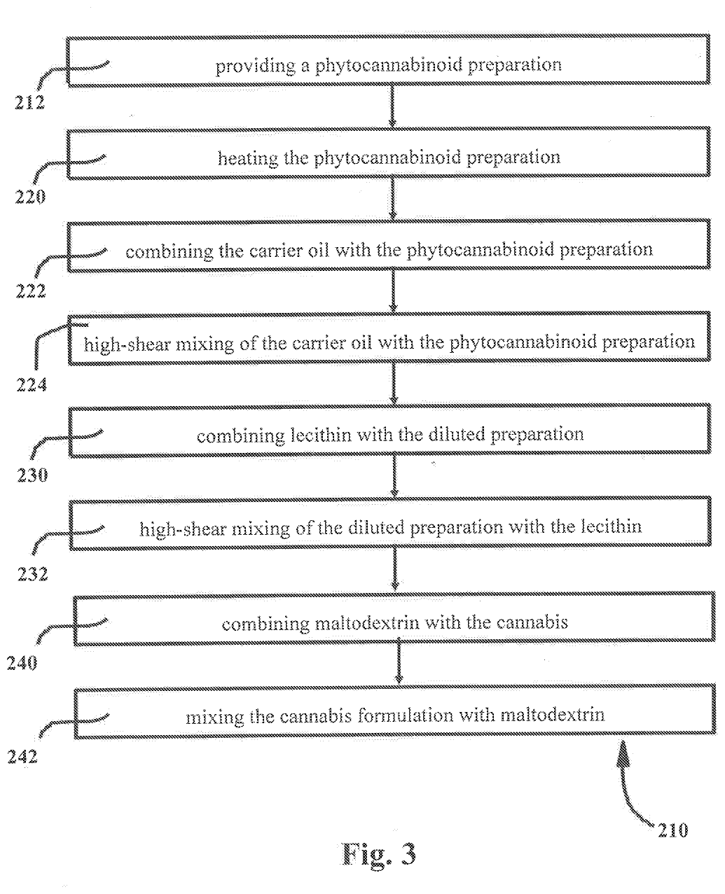

FIG. 3 shows a method of preparing a phytocannabinoid formulation 210. The method 210 includes providing a phytocannabinoid preparation 212. Heating the phytocannabinoid preparation 220 follows until the phytocannabinoid preparation is flowable. After heating the phytocannabinoid preparation 220, combining the carrier oil with the phytocannabinoid preparation 222 follows. After combining the carrier oil with the phytocannabinoid preparation 222, high-shear mixing of the carrier oil with the phytocannabinoid preparation 224 follows until the diluted preparation is produced by the high-shear mixing. Combining lecithin with the diluted preparation 230 follows preparation of the diluted preparation. High-shear mixing 232 of the diluted preparation with the lecithin follows to prepare the cannabis formulation. Combining maltodextrin with the cannabis formulation 240 and mixing of the cannabis formulation with the maltodextrin 242 follows until the solid preparation is produced by the high-shear mixing.

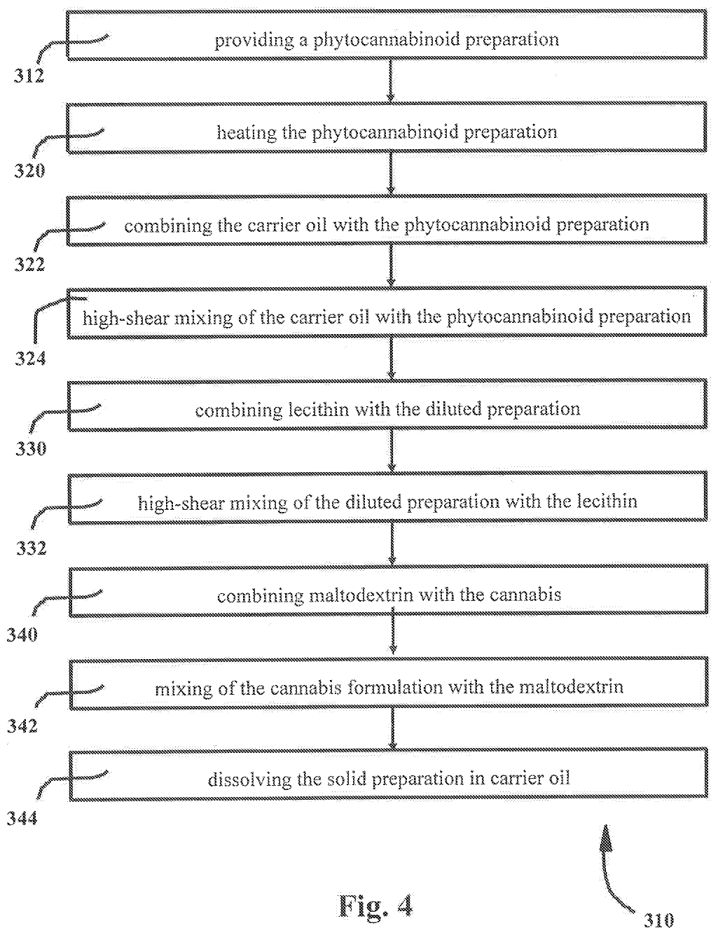

FIG. 4 shows a method of preparing a phytocannabinoid formulation 310. The method 310 includes providing a phytocannabinoid preparation 312. Heating the phytocannabinoid preparation 320 follows until the phytocannabinoid preparation is flowable. After heating the phytocannabinoid preparation 320, combining the carrier oil with the phytocannabinoid preparation 322 follows. After combining the carrier oil with the phytocannabinoid preparation 322, high-shear mixing of the carrier oil with the phytocannabinoid preparation 324 follows until the diluted preparation is produced by the high-shear mixing. Combining lecithin with the diluted preparation 330 follows preparation of the diluted preparation. High-shear mixing 332 of the diluted preparation with the lecithin follows to prepare the cannabis formulation. Combining the maltodextrin with the cannabis formulation 340 and mixing of the cannabis formulation with the maltodextrin 342 follows until the solid preparation is produced by the high-shear mixing. Dissolving the solid preparation in carrier oil 344 may follow mixing of the cannabis formulation with the maltodextrin 342 to provide a liquid formulation.

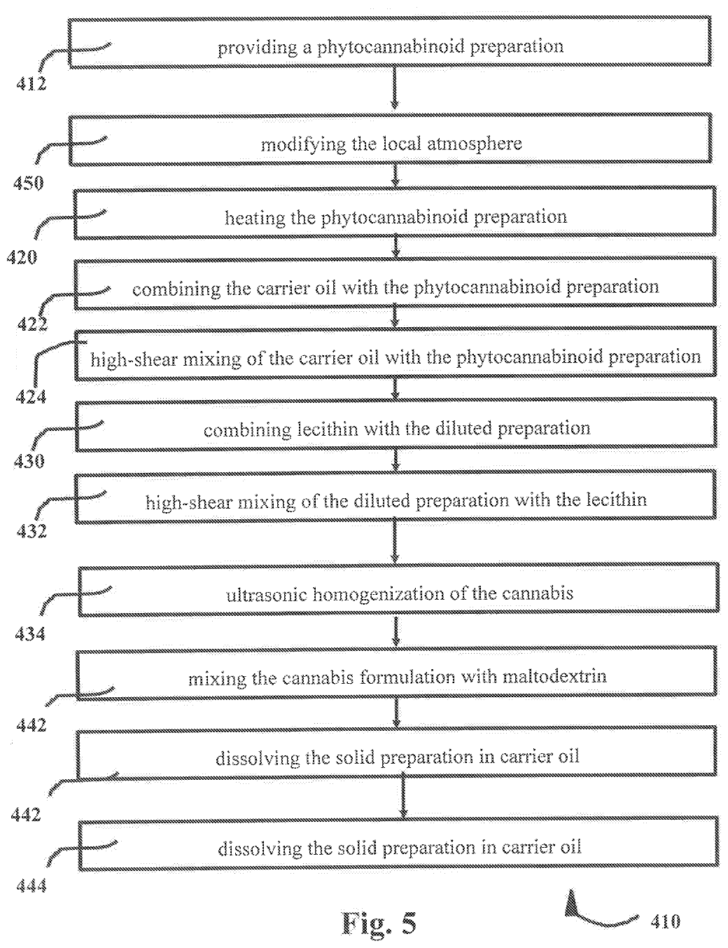

FIG. 5 shows a method of preparing a phytocannabinoid formulation 410 under nitrogen, argon or another gas that is protective from oxidative damage. The method 410 includes providing a phytocannabinoid preparation 412. Heating the phytocannabinoid preparation 420 follows until the phytocannabinoid preparation is flowable. Modifying the local atmosphere 450 follows by exposing the phytocannabinoid preparation first to vacuum then to nitrogen, argon or another inert gas that is protective from oxidative damage. The subsequent steps of the method 410 take place in the modified local atmosphere.

After heating the phytocannabinoid preparation 420 and modifying the local atmosphere 450, combining the carrier oil with the phytocannabinoid preparation 422 follows. After combining the carrier oil with the phytocannabinoid preparation 422, high-shear mixing of the carrier oil with the phytocannabinoid preparation 424 follows until the diluted preparation is produced by the high-shear mixing. Combining lecithin with the diluted preparation 430 follows preparation of the diluted preparation. High-shear mixing 432 of the diluted preparation with the lecithin follows to prepare the cannabis formulation. Ultrasonic homogenization of the cannabis formulation 434 follows, resulting in the cavitated formulation. Combining the maltodextrin with the cannabis formulation 440 and mixing of the cannabis formulation with the maltodextrin 442 follows until the solid preparation is produced by the high-shear mixing. Packaging under the modified local atmosphere 452 follows mixing of the cannabis formulation with the maltodextrin 442.

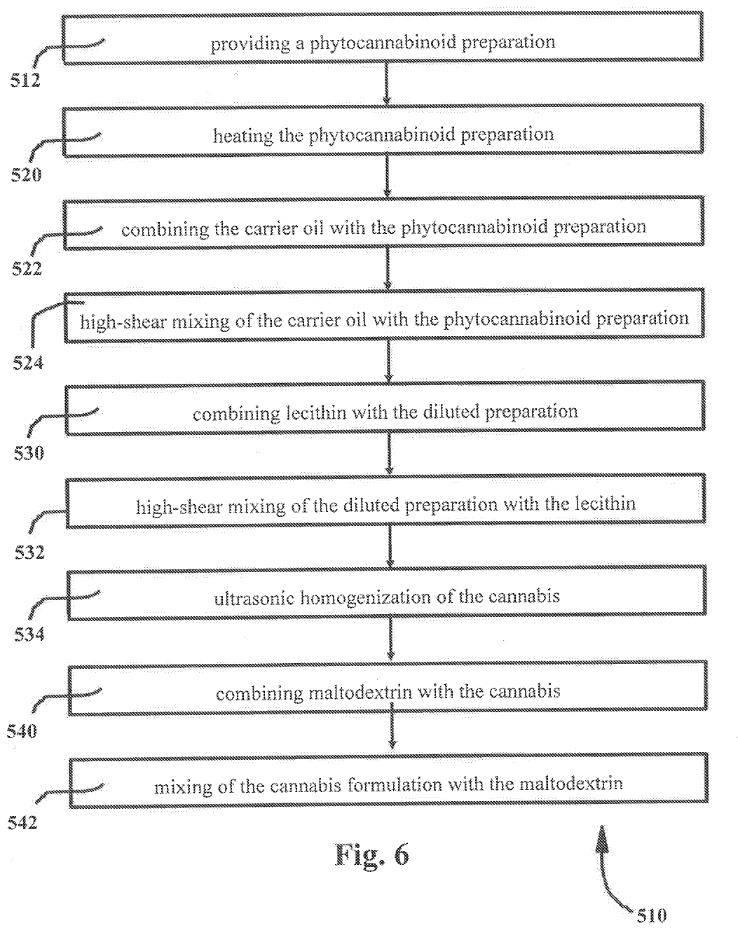

FIG. 6 shows a method of preparing a phytocannabinoid formulation 510. The method 510 includes providing a phytocannabinoid preparation 512. Heating the phytocannabinoid preparation 520 follows until the phytocannabinoid preparation is flowable. After heating the phytocannabinoid preparation 520, combining the carrier oil with the phytocannabinoid preparation 522 follows. After combining the carrier oil with the phytocannabinoid preparation 522, high-shear mixing of the carrier oil with the phytocannabinoid preparation 524 follows until the diluted preparation is produced by the high-shear mixing. Combining lecithin with the diluted preparation 530 follows preparation of the diluted preparation. High-shear mixing 532 of the diluted preparation with the lecithin follows to prepare the cannabis formulation. Ultrasonic homogenization of the cannabis formulation 534 follows, resulting in the cavitated formulation. Combining the maltodextrin with the cannabis formulation 540 and mixing of the cannabis formulation with the maltodextrin 542 follows until the solid preparation is produced by the high-shear mixing.

### EXAMPLE

A phytocannabinoid preparation including 50 grams of cannabis resin was extracted using passive closed loop ethanol . The sample included approximately 35 grams of THC and 2.5 grams of CBD. The samples was placed in a 1 L glass beaker. The glass beaker was placed in a water bath and heated to a setpoint temperature of 102° F. A portion of 7 ml was drawn off the heated phytocannabinoid preparation and placed into a warmed Gilmont falling ball Viscometer with a No. 3 Tantalum ball. The viscosity was measured at a dynamic viscosity of 1,600 centipoise. Coconut oil with a 76° F. melting point was warmed to 102° F. and poured into the phytocannabinoid preparation. The lower the dynamic viscosity of the phytocannabinoid preparation, the less carrier oil that will be necessary to prepare the diluted preparation by high-shear mixing of the phytocannabinoid preparation and the carrier oil. While maintaining the setpoint temperature, the phytocannabinoid preparation was mixed with the carrier oil by high-shear mixing using a handheld rotor-stator mixer at 35,000 revolutions per minute. After high-shear mechanical mixing is completed, the water bath and sample temperatures stabilized. Lecithin heated to 102° F. was added to the diluted preparation in a ratio of 1:2 lecithin:diluted preparation. High-shear mixing was resumed on the phytocannabinoid preparation, carrier oil and Lecithin.

proceed to adding lecithin.

In the preceding description, for purposes of explanation, numerous details are set forth in order to provide a thorough understanding of the embodiments. However, it will be apparent to one skilled in the art that these specific details are not required.

The above-described embodiments are intended to be examples only. Alterations, modifications and variations can be effected to the particular embodiments by those of skill in the art without departing from the scope, which is defined solely by the claims appended hereto.

What is claimed is:

1. A method of preparing a cannabis formulation comprising:
providing a phytocannabinoid preparation;
heating the phytocannabinoid preparation to a setpoint temperature at which the phytocannabinoid preparation is flowable;
combining a carrier oil with the phytocannabinoid preparation;
high-shear mixing the carrier oil with the phytocannabinoid preparation to provide a diluted preparation;
combining lecithin with the diluted preparation; and
high-shear mixing the lecithin with the diluted preparation, providing the cannabis formulation.

2. The method of claim 1 wherein the phytocannabinoid preparation comprises a phytocannabinoid distillate.

3. The method of claim 1 wherein the phytocannabinoid preparation comprises terpenoids.

4. The method of claim 3 wherein the phytocannabinoid preparation comprises phytocannabinoids and terpenoids extracted from a single biomass source.

5. The method of claim 4 wherein the single biomass source comprises plant matter from a single clonal strain or stable variety of Cannabis sativa.

6. The method of claim 1 wherein the setpoint temperature is between 90 and 105° F.

7. The method of claim 1 wherein the cannabis preparation has a dynamic viscosity of between 1,500 and 2,000 centipoise at the setpoint temperature.

8. The method of claim 1 wherein combining the carrier oil with the cannabis preparation comprises heating the carrier oil to the setpoint temperature.

9. The method of claim 1 wherein high-shear mixing the carrier oil with the phytocannabinoid preparation comprises high-shear mixing with a rotor-stator mixer.

10. The method of claim 1 wherein high-shear mixing the carrier oil with the phytocannabinoid preparation comprises mixing at the setpoint temperature.

11. The method of claim 1 wherein combining lecithin with the diluted preparation comprises heating the lecithin to the setpoint temperature.

12. The method of claim 1 wherein high-shear mixing the lecithin with the diluted preparation comprises heating the lecithin to a lecithin setpoint temperature.

13. The method of claim 1 wherein high-shear mixing the lecithin with the diluted preparation comprises high-shear mixing with a rotor-stator mixer.

14. The method of claim 1 wherein the lecithin comprises sunflower lecithin.

15. The method of claim 1 further comprising applying ultrasonic homogenization to the cannabis formulation, providing a cavitated cannabis formulation.

16. The method of claim 1 further comprising combining maltodextrin with the cannabis formulation, providing a solid cannabis formulation.

17. The method of claim 16 wherein combining maltodextrin with the cannabis formulation comprises maintaining the cannabis formulation at a mixing temperature at which the cannabis formulation has a dynamic viscosity of between 150 and 400 centipoise.

18. The method of claim 17 wherein the mixing temperature is between 80 and 85 degrees Fahrenheit.

19. The method of claim 16 wherein combining maltodextrin with the cannabis formulation comprises adding an additional carrier oil to the cannabis

20. The method of claim 16 further comprising dehydrating the maltodextrin.

21. The method of claim 1 further comprising combining an antioxidant with the cannabis formulation.

22. The method of claim 21 wherein the antioxidant comprises five percent or less by volume in the cannabis formulation Vitamin E.

23. The method of claim 1 further comprising combining an additional carrier oil to the cannabis formulation, targeting a phytocannabinoid concentration to provide a calibrated liquid cannabis formulation.

24. The method of claim 1 wherein providing the phytocannabinoid preparation, heating the phytocannabinoid preparation to the setpoint temperature, combining the carrier oil with the phytocannabinoid preparation, high-shear mixing the carrier oil with the phytocannabinoid preparation, combining lecithin with the diluted preparation, and high-shear mixing the lecithin with the diluted preparation are performed in a nitrogen-enriched atmosphere for dehydrating the phytocannabinoid preparation and mitigating oxidative damage.

25. The method of claim 24 further comprising packaging the phytocannabinoid preparation in the nitrogen-enriched atmosphere.

26. The method of claim 24 further comprising combining maltodextrin with the cannabis formulation in the nitrogen-enriched atmosphere, providing a solid cannabis formulation.

27. The method of claim 1 wherein providing the phytocannabinoid preparation, heating the phytocannabinoid preparation to the setpoint temperature, combining the carrier oil with the phytocannabinoid preparation, high-shear mixing the carrier oil with the phytocannabinoid preparation, combining lecithin with the diluted preparation, and high-shear mixing the lecithin with the diluted preparation are performed in a argon-enriched atmosphere for dehydrating the phytocannabinoid preparation and mitigating oxidative damage.

28. The method of claim 27 further comprising packaging the phytocannabinoid preparation in the argon-enriched atmosphere.

29. The method of claim 27 further comprising combining maltodextrin with the cannabis formulation in the argon-enriched atmosphere, providing a solid cannabis formulation.

30. A phytocannabinoid formulation prepared according to the method of claim 1.

31. A method of preparing a comprising:
providing a phytocannabinoid preparation;
heating the phytocannabinoid preparation to a setpoint temperature at which the phytocannabinoid preparation has a dynamic viscosity of between 1,500 and 2,000 centipoise;
combining a carrier oil with the phytocannabinoid preparation;
high-shear mixing the carrier oil with the phytocannabinoid preparation at or above the setpoint temperature to provide a diluted preparation;
heating lecithin to provide liquid lecithin;
combining the liquid lecithin with the diluted preparation;
high-shear mixing the liquid lecithin with the diluted preparation, providing the cannabis formulation; and
applying ultrasonic homogenization to the cannabis formulation, providing cavitated cannabis formulation.

32. The method of claim 31 further comprising combining maltodextrin with the cannabis formulation, providing a solid cannabis formulation.

33. The method of claim 32 wherein combining maltodextrin with the cannabis formulation comprises maintaining the cannabis formulation at a mixing temperature at which the cannabis formulation has a dynamic viscosity of between 150 and 400 centipoise.

34. The method of claim 33 wherein the mixing temperature is between 80 and 85 degrees Fahrenheit.

35. The method of claim 32 wherein combining maltodextrin with the cannabis formulation comprises adding an additional carrier oil to the cannabis

36. The method of claim 32 further comprising dehydrating the maltodextrin.

37. A phytocannabinoid formulation prepared according to the method of claim 31.
